(19) United States
(12) Patent Application Publication
(10) Pub. No.: US 2025/0001088 A1
(43) Pub. Date: Jan. 2, 2025

(54) THERAPEUTIC AGENT DELIVERY DEVICE WITH SINGLE MOTOR DRIVE

(71) Applicant: Eli Lilly and Company, Indianapolis, IN (US)

(72) Inventors: David Arthur HOLLEY, Lancaster, OH (US); Michael Lewis MARSHALL, Lewis Center, OH (US)

(21) Appl. No.: 18/263,083

(22) PCT Filed: Jan. 31, 2023
(86) PCT No.: PCT/US2023/011918

§ 371 Date: Jul. 26, 2023

Related U.S. Application Data
(60) Provisional application No. 63/306,130, filed on Feb. 3, 2022.

(51) Int. Cl. A61M 5/315; A61M 5/20; A61M 5/24

(52) U.S. Cl. CPC A61M 5/31585; A61M 2005/206; A61M 2005/2411

(57) ABSTRACT

A therapeutic agent delivery device includes a syringe assembly having a plunger, a drive mechanism, and a shuttle system coupled to a syringe of the syringe assembly. The drive mechanism includes a motor and a lead screw to engage the plunger, and a controller configured to facilitate movement of the shuttle system distally relative to the syringe assembly and movement of the syringe distally to extend a needle from the therapeutic agent delivery device. The lead screw can drive the plunger for drug delivery. Rotation of the lead screw after the plunger is driven can facilitate retraction of the lead screw from the plunger and facilitate movement of the shuttle system proximally and the syringe proximally relative to the syringe assembly. The drive mechanism and the shuttle system may be an integrated assembly.

### CROSS-REFERENCE TO RELATED APPLICATION

This application claims the benefit of Provisional Application No. 63/306,130 filed Feb. 3, 2022, which is incorporated by reference herein in its entirety.

### BACKGROUND

The present disclosure pertains to therapeutic agent delivery devices, and, in particular, to a portable therapeutic agent delivery device such as an injector pen.

Patients suffering from a number of different diseases must frequently inject themselves with medication. To allow a person to conveniently and accurately self-administer medicine, a variety of devices broadly known as injector pens or injection pens have been developed. Generally, these pens are equipped with a cartridge including a piston and one or more doses of liquid medication. Typically, a drive member, extending from within a base of the injector pen and operably connected with rearward mechanisms of the pen that control drive member motion, is movable forward to advance the piston in the cartridge in such a manner as to dispense the contained medication from an outlet at the opposite cartridge end, the outlet typically a needle that penetrates a plunger at that opposite end. In disposable pens, once a pen has been used and exhausted the supply of medication within the cartridge, the entire pen is discarded by a user, who may then begin using a replacement pen. In reusable pens, after a pen has been used and exhausted the supply of medication within the cartridge, the pen is disassembled, the spent cartridge is replaced with a fresh cartridge, and the pen is reassembled for its subsequent use.

It would be desirable to provide a therapeutic agent delivery device with improved features for providing a reusable device while simplifying the manufacture of the therapeutic agent delivery device.

### SUMMARY

According to an example (“Example 1”) of the present disclosure, a therapeutic agent delivery device having a proximal end and a distal end is described. The device includes a syringe assembly including a syringe having a plunger, a shuttle system coupled to the syringe, and a drive mechanism including a lead screw. The lead screw has a distal end configured to engage the plunger. A controller is configured to: activate the drive mechanism to rotate the lead screw in a first direction to a position to facilitate movement of the shuttle system distally relative to the syringe assembly and movement of the syringe distally relative to the syringe assembly from a first position to a second position to extend a needle from the distal end of the therapeutic agent delivery device; activate the drive mechanism to rotate the lead screw in a second direction, the distal end of the lead screw driving the plunger of the syringe in the second position for therapeutic agent delivery; activate the drive mechanism to rotate the lead screw in the first direction after rotation of the said lead screw in the second direction to retract the distal end of the lead screw from the plunger and to facilitate movement of the shuttle system proximally and the syringe proximally relative to the syringe assembly from the second position toward the first position; and end activation of the drive mechanism to stop rotation of the lead screw after proximal movement of the shuttle system and the syringe.

According to an example (“Example 2”) of the present disclosure, a plunger drive and syringe movement system is described. The system includes a cam follower, a cam ramp, a one-way clutch coupled to the cam follower, and a carriage coupled to a syringe of a syringe assembly. The system includes a lead screw that is operatively coupled to the cam follower and a motor. The lead screw includes a distal end to engage a plunger within the syringe. The motor is operatively coupled to the lead screw to drive the lead screw in a first direction and a second direction. A drive of the lead screw in the first direction facilitates rotation of the cam follower to a position relative to the cam ramp to permit a displacement change to the carriage relative to the cam ramp. The drive of the lead screw in the second direction facilitates movement of the plunger distally to expel a therapeutic agent contained within the syringe.

According to an example (“Example 3”) of the present disclosure, a method for delivering a therapeutic agent from a therapeutic agent delivery device is provided. The therapeutic agent delivery device has a reusable assembly and a syringe assembly. The method includes one or more of the steps: Providing the therapeutic agent delivery device in a rest configuration defined by a cam follower of the device being positioned in a first position on a plateau portion of a cam ramp. Rotating the cam follower relative to the cam ramp to a second position within a valley portion of the cam ramp and thus displacing a shuttle assembly of the device and driving a syringe of said syringe assembly distally relative to the therapeutic agent delivery device to a deployed configuration. Driving a lead screw distally along a longitudinal axis of the therapeutic delivery device, such that the lead screw displaces a plunger of the syringe in the deployed configuration to dispense a therapeutic agent.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a top perspective view of a therapeutic agent delivery device according to an embodiment of the present disclosure.

FIG. 2 is a front perspective view of the therapeutic agent delivery device of FIG. 1.

FIG. 3 is a top perspective view of a syringe assembly portion of the therapeutic agent delivery device of FIG. 1;

FIG. 4 is an exploded view of the therapeutic agent delivery device of FIG. 1.

FIG. 5 is a top perspective view of various internal components of the therapeutic agent delivery device of FIG. 1.

FIG. 6 is a cross-sectional view of the internal components of the therapeutic agent delivery device of FIG. 5.

FIG. 7 is a schematic representation of an electronics assembly of the therapeutic agent delivery device of FIG. 1.

FIG. 8 is a transverse sectional view of a drive mechanism of the therapeutic agent delivery device of FIG. 1

FIG. 9A is a top perspective view of a portion of the drive mechanism of the therapeutic agent delivery device of FIG. 1.

FIG. 9B is a transverse sectional view of the portion of the drive mechanism shown in FIG. 9A.

FIG. 10A is a top perspective view of a portion of the drive mechanism of the therapeutic agent delivery device of FIG. 1.

FIG. 10B is a transverse sectional view of the portion of the drive mechanism of FIG. 10A.

FIG. 11A is a top perspective view of a portion of the therapeutic delivery device of FIG. 1.

FIG. 11B is a sectional view of the portion of the therapeutic delivery device shown in FIG. 11A.

FIG. 12A is a transverse sectional view of the drive mechanism of the therapeutic delivery device in a first position.

FIG. 12B is a transverse sectional view of the drive mechanism of the therapeutic delivery device in a second position.

FIGS. 13A-13F illustrate a transverse sectional view of the therapeutic delivery device throughout various steps of delivering the therapeutic.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION

Therapeutic agent delivery devices according to the present disclosure carry and dispense one or more therapeutic agents, which may also be referred to as medications or drugs. Such therapeutic agents may include, for example, epinephrine, anaesthetics, analgesics, steroids, insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, combined GIP/GLP-1 agonists such as tirzepatide, basal insulins, oxyntomodulin analogs, oxyntomodulin derivatives, therapeutic antibodies including but not limited to IL-23 antibody analogs or derivatives, such as mirikizumab, IL-17 antibody analogs or derivatives, such as ixekizumab, therapeutic agents for pain-related treatments, such as galcanzeumab or lasmiditan, or lebrikizumab and any therapeutic agent that is capable of delivery by the devices described herein. Therapeutic agent delivery devices according to the present disclosure are operated in a manner generally as described herein by a user (for example, a healthcare professional, a caregiver, or another person) to deliver one or more therapeutic agents to a patient (for example, another person or the user).

Any directional references used with respect to any of the Figures, such as right or left, up or down, or top or bottom, are intended for convenience of description, and does not limit the present disclosure or any of its components to any particular positional or spatial orientation. Rotation may be described herein in terms of the clockwise or counterclockwise direction, and it is noted that the rotation should not be limited to the specific direction described as the mechanical components can be arranged to function when specific components are rotating a direction opposite. To this end, rotation in a first direction or a second direction may be used to refer to either the clockwise or counterclockwise direction.

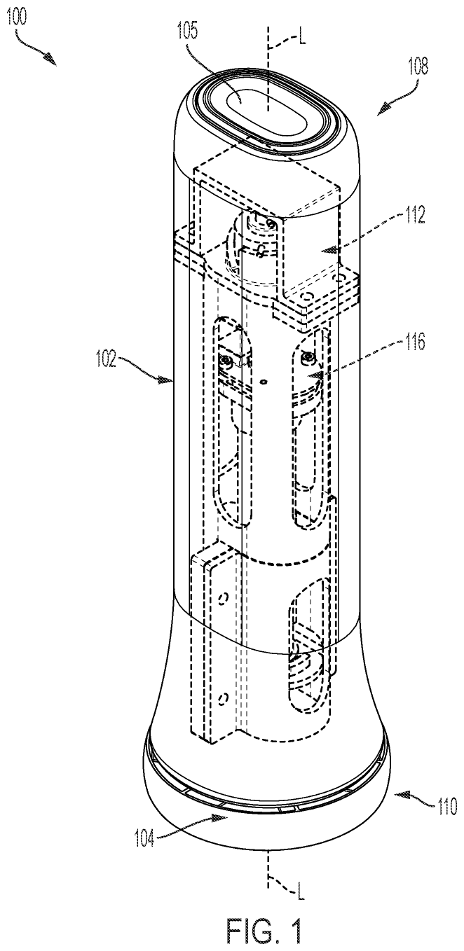

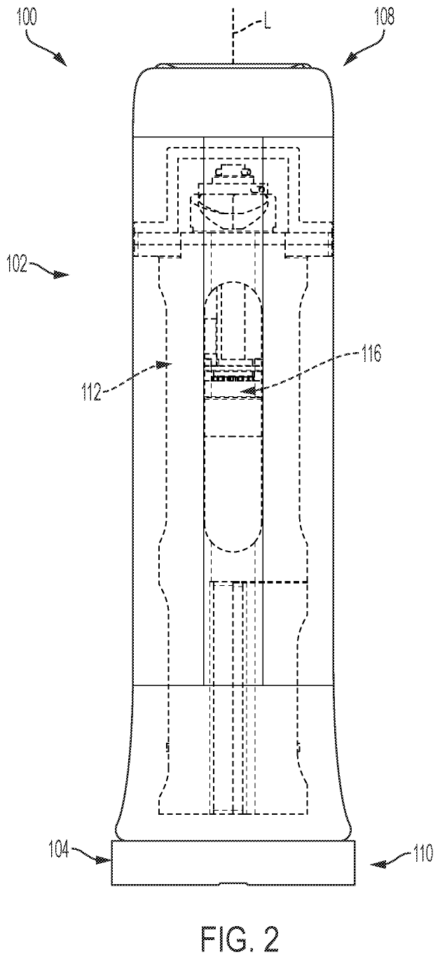

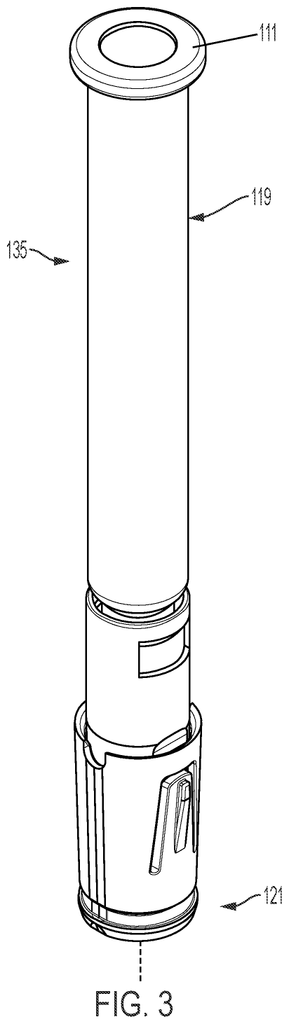

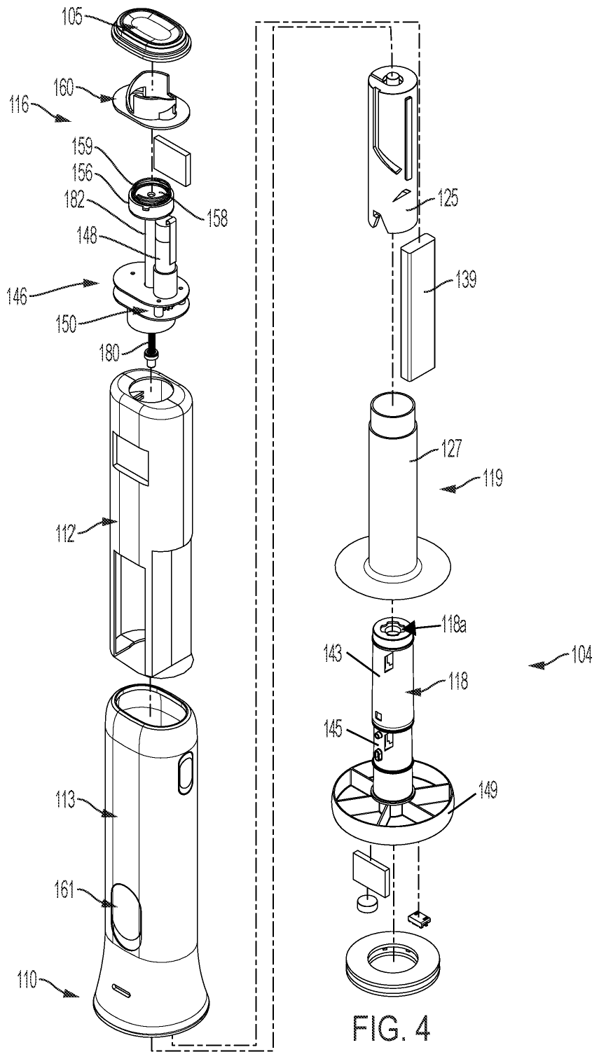

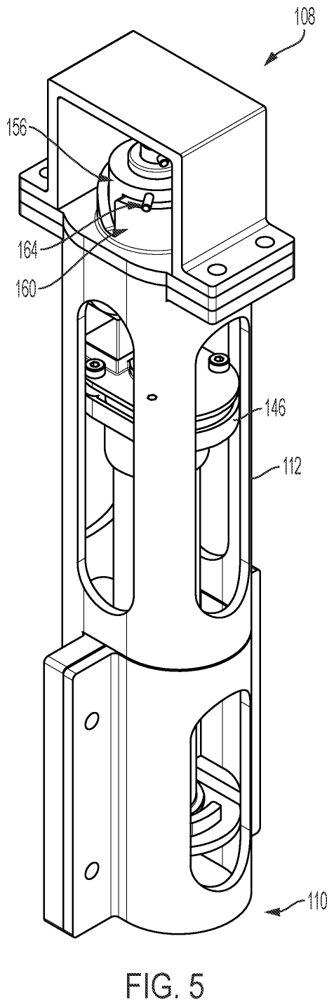

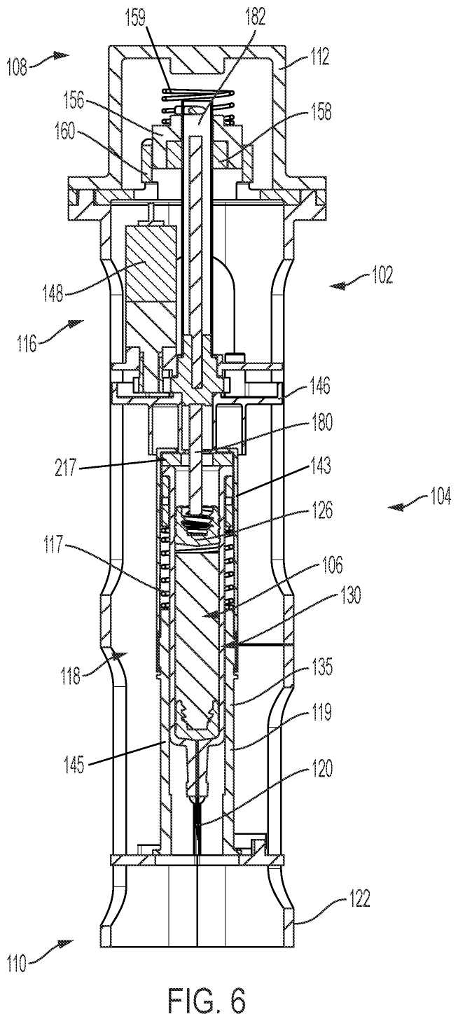

FIGS. 1-2 illustrate a therapeutic agent delivery device 100 according to an exemplary embodiment of the present disclosure. Illustratively, the therapeutic agent delivery device 100 has an injector pen-like shape, although other shapes may alternatively be used. The therapeutic agent delivery device 100 generally includes a reusable portion 102, which may also be referred to as a drive portion, and a disposable portion 104, which may also be referred to as a drug carrying portion or cartridge. The reusable portion 102 facilitates delivery of a therapeutic agent 106 (FIG. 6). In addition, the disposable portion 104 detachably couples to the reusable portion 102 such that after the therapeutic agent 106 has been delivered from the disposable portion 104, the disposable portion 104 may be detached from the reusable portion 102 and discarded. Another disposable portion (not shown—for example, having the same or different features than the disposable portion 104) may then be attached to the reusable portion 102, and the therapeutic agent delivery device 100 is thereby ready for subsequent use. A portion of the disposable portion 104 will be described further with reference to FIG. 3. The therapeutic agent delivery device 100 includes a proximal end 108 and a distal end 110 opposite the proximal end 108. During use of the therapeutic agent delivery device 100, the proximal end 108 would be further from the patient and configured to be actuated by the user, and the distal end 110 would be closer to the patient and configured to deliver the therapeutic agent to the patient. The therapeutic agent delivery device 100 also includes a longitudinal axis L extending between the proximal end 108 and the distal end 110. FIGS. 1-2 additionally illustrate various internal components of the therapeutic agent delivery device 100 in phantom. For example, a reusable portion housing 112 internal to the portion 102 having components of a drive mechanism 116 and portions of a syringe assembly 118 (FIG. 3) are shown in phantom within the therapeutic delivery device 100, as will be described further with reference to FIGS. 4-5.

FIG. 3 illustrates a perspective view of an example embodiment of a portion of the disposable portion 104 of the therapeutic agent delivery device 100. More specifically, FIG. 3 illustrates a syringe 119 of the syringe assembly 118 (FIG. 4). The syringe assembly 118 includes a rigid needle protection portion at a distal end 121 of the syringe 119 to shield a needle, or other carrier for the therapeutic agent, before deployment of the syringe assembly 118. In this way, during the use of the therapeutic agent delivery device 100, the patient, physician, or user may deploy the needle, or other carrier for the agent, within the syringe assembly 118 to deliver a therapeutic agent and the disposable portion 104 may be disposed and a new disposable portion 104 may be replaced within a reusable portion 102. However, various other embodiments of the disposable portion 104 may be incorporated with the use of the reusable portion 102.

The above-described configuration of the therapeutic agent delivery device 100 provides the advantage in that a portion, namely the reusable portion 102, may house circuitry or other components that may be more expensive and be maintained for use. For example, the therapeutic agent delivery device 100 may be a delivery device for injecting insulin or other diabetes therapeutics. The patient may inject the therapeutic agent from the disposable portion 104 of the device 100, the disposable portion 104 disposed and removed from the therapeutic agent delivery device 100, and the reusable portion 102 maintained for further use. This may reduce not only costs for the manufacturer, but also for the patient or the user. However, in various other embodiments, the entirety of the therapeutic agent delivery device 100 may be disposable and meant only for one time use. The therapeutic agent delivery device 100 and its internal components will be described further herein.

With specific reference to the exploded view of FIG. 4, the various components of the therapeutic agent delivery device 100 will be described. As illustrated, at the proximal end 108 of the device 100, the device 100 includes an outer frame 113. As previously described, the device 100, and thus the outer frame 113, may have a pen-like shape. The outer frame 113 includes a user ejector input 161 for ejecting the syringe assembly of the disposable portion 104 when needed, such as, for example, after the delivery of the therapeutic agent. While illustrated as positioned on a side wall of the outer frame 113, the user ejector input 161 may have various other configurations, for example on a proximalmost surface of the outer frame 113 or on a varying side of the outer frame 113. Further, the housing 112 of the reusable portion 102 is an internal frame that may be received within the outer frame 113 of the device 100. More specifically, the outer frame 113 may be generally cylindrical in shape and define an interior region for receiving the housing 112 and various internal components of the reusable portion 102.

With reference to FIG. 4 and FIG. 6, the syringe assembly 118 includes an assembly housing 141 that surrounds the syringe 119. The assembly housing 141 may includes a proximal portion 143 and a distal portion 145 that couple to one another. The proximal portion 143 is a tubular body sized to fit over the flange 111 and barrel 135 of the syringe 119. The distal portion 145 is a tubular body sized to fit over the barrel 135 of the syringe 119. The proximal portion 143 is shown to have a distal end opening sized to receive the proximal end of the distal portion 145 to define an enclosed housing for the syringe. A base cap 149 is removably coupled to the distal end of the distal portion 145. The base cap 149 is configured to be withdrawn by the user prior to use of the device. When withdrawn, the base cap 149 that is secured to the rigid needle protection portion at the distal end 121 of the syringe 119 is configured to remove simultaneously the rigid needle protection portion from a needle 120, thereby exposing the needle for use. A retraction biasing member 117, shown in FIG. 6 in its expanded configuration, is positioned between the proximal facing edge of the distal portion 145 and the flange 111 of the syringe 119 in the annular space defined between the interior of the proximal portion 143 and the outer surface of the syringe barrel 135.

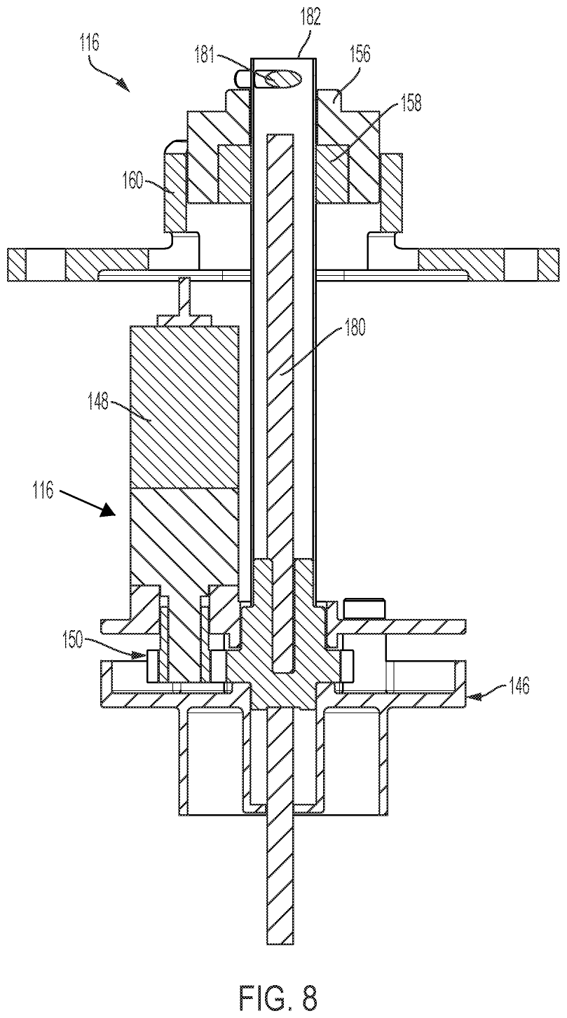

The reusuable portion 102 comprises a shuttle system 109 (see FIG. 8). In one embodiment, the shuttle system 109 is coupled to the syringe assembly 118 and is configured to move the syringe 119 relative to the syringe assembly 118 and the housing 112 at least in the distal direction and may be configured to move the syringe 119 also in the proximal direction. The shuttle system 109 may include a carriage 146, a coupling tube 182, a clutch 158 that receives an insertion biasing member 159, a cam follower 156 coupled to the clutch 158, and a guide housing, also referred to as a guide 160, positioned radially relative to the cam follower 156.

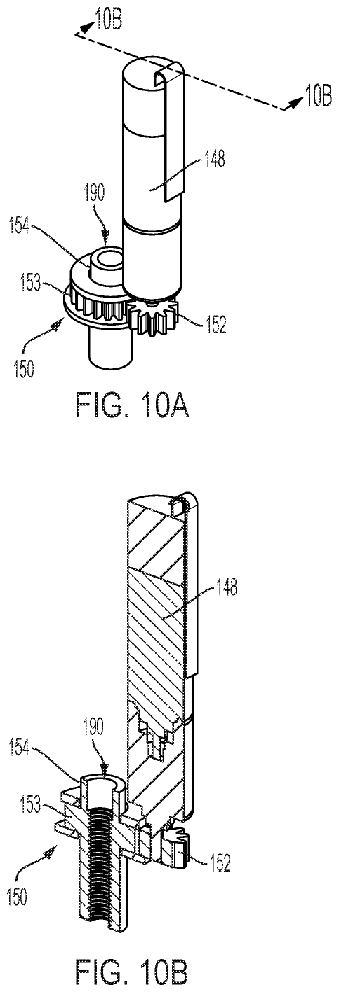

The reusable portion 102 comprises the drive mechanism 116, portions of which are shown in FIGS. 10A-10B. In one embodiment the drive mechanism includes a gear train 150 positioned within the carriage 146, a lead screw 180 and at least one actuator 148 extending from the carriage 146, and a nut 154. Actuator includes a drive shaft that is controllably rotated by input from the controller. As will be described, activation of the drive mechanism can cause the lead screw to move in the proximal direction or in the distal direction.

Although the shuttle system 109 and the drive mechanism 116 can be different assemblies coupled to the housing, they may also be integrated into a single subassembly, for example, as shown in FIG. 8. To this end, at times the description herein may refer to components of the drive mechanism 116 and the shuttle system 109 as belong to either one. For example, the carriage 146 may also referred to herein as a motor shuttle since the actuator is mounted to the carriage, the gear train 150 and the nut 154 can be positioned within the carriage 146, and the lead screw 180 extends through the carriage 146 and within the coupling tube 182. The user input 105 may be coupled with the outer frame 113 at the proximal end to enclose the drive mechanism 116 components within the outer frame 113, and thus the device 100. Input 105 may be a movable button or a touch/pressure surface to start actuation.

FIG. 4 shows another embodiment of the housing, referred to as housing 112′. The housing 112 or 112′ additionally receives a power supply 138, shown as at least one battery 139, that may be positioned adjacent a syringe assembly carrier 129 that is coupled to the housing 112′. The syringe assembly carrier 129 and a syringe carrier housing 125 that are coupled to housing 112′ are together configured to house the syringe assembly 118. The syringe carrier housing 125 and carrier 129 can be permanently attached to the housing 112. When inserted into the housing 112′, the syringe assembly 118 remains axially and rotationally fixed during operation of the drive mechanism 116. After use, the user may remove the syringe assembly 118 from the housing 112′ for disposal. Syringe carrier housing 125 may be coupled to the proximal end of the syringe assembly carrier 129, covering the syringe assembly 118, except for the base cap 149.

Further, as illustrated in FIG. 4, the syringe assembly carrier 129 may comprise a printed circuit board 127 disposed around the shaft of the syringe assembly carrier 129 and in some embodiments, the syringe assembly carrier 129 may be coupled with components of the an electronics assembly, such as, for example, an audio speaker and a charging port used to plug in an energy source to charge the battery 139. Board 127 contains one or more the components of an electronics assembly 134, described below. Other portions of the device 100 may include additional circuit boards and electronic assembly components. Further, the device 100 includes the disposable portion 104, illustratively a disposable syringe assembly 118. The syringe assembly 118 may be received within the device frame 113, such as, for example, via the syringe carrier 129 and, as will be described further herein, the plunger of the syringe assembly 118 is positioned to be driven by the drive mechanism 116. The shuttle system 109 and the drive mechanism 116 are configured (1) to drive the syringe 119 relative to the assembly housing 141 of the syringe assembly 118 and relative to the housing 112 or 112′ and frame 113 of the device and (2) to move the plunger to expel the therapeutic agent 106 (FIG. 6). As described, the syringe assembly 118 as the disposable portion 104 may then be removed and replaced with a separate and/or new disposable syringe assembly. In some embodiments, the device in its entirety may be disposable and the syringe assembly 118 is permanently associated with the device and cannot be withdrawn.

With reference now to the perspective view of FIG. 5 and the transverse sectional view of FIG. 6, internal components and other features of the reusable portion 102 and the disposable portion 104 are illustrated and described further. FIGS. 5-6 shows another embodiment of the housing of the reusable portion 102, referred here to housing 112, that moveably carries the user input 105 (FIGS. 1 and 4) through the outer frame 113 (FIG. 4) and the drive mechanism 116. The user input 105 may be received via mechanical and/or electrical means, for example a mechanical switch or an electrical switch. The user input 105 may be actuable (for example, slidable or depressible) by a user to actuate the drive mechanism 116. The shuttle system 109 and the drive mechanism 116 thereby translates distally in concert relative to the housing 112 or 112′ to drive the syringe 119 relative to the syringe assembly housing of the disposable portion 104. More specifically, the shuttle system 109 and the drive mechanism 116 translatably drives the syringe 119 relative to the syringe assembly 118 from a stowed configuration to a deployed configuration. In the stowed configuration, the needle 120 of the syringe 119 is disposed proximally relative to a distal end 122 of the housing 112 or 112′ of the device. In other words, in the stowed configuration the needle 120 is retracted within the therapeutic agent delivery device 100. In the deployed configuration, the needle 120 at least partially extends distally beyond the distal end 122 of the housing 112 or 112′. As a result, in the deployed configuration, the needle 120 is configured to pierce the skin of a patient when the distal end of the device in in contact with a patient's skin for subcutaneously delivery of the therapeutic agent. During the deployed configuration, the drive mechanism 116 is activated to distally translate a plunger 126 of the syringe 119 relative to a syringe barrel 135 of the syringe 119. The plunger 126, carried in a therapeutic agent-carrying passageway 130 of the syringe 119, is driven distally to cause the therapeutic agent 106 to be discharged via the needle 120. As illustrated in FIG. 6, components of the syringe carrier housing 125 and the syringe assembly carrier 129 are missing, leaving the syringe assembly 118, that would be inside the syringe carrier housing 125 and carrier 129 of FIG. 4. The retraction biasing member 117 is shown in its expanded configuration can be loaded during the movement of the syringe 119 relative to the syringe assembly 118. The retraction biasing member 117 may be a spring, however various other biasing members may be incorporated. When the syringe 119 moves axially relative to the syringe assembly housing 141, a portion of the syringe 119, for example the flange 111, will engage the retraction biasing member 117, loading the retraction biasing member 117 in the compressed configuration for later deployment to aid in driving the retraction of the syringe 119 relative the assembly housing 141. Additional details of these components are described below. The retraction of the syringe 119 and the lead screw 180 will be described below.

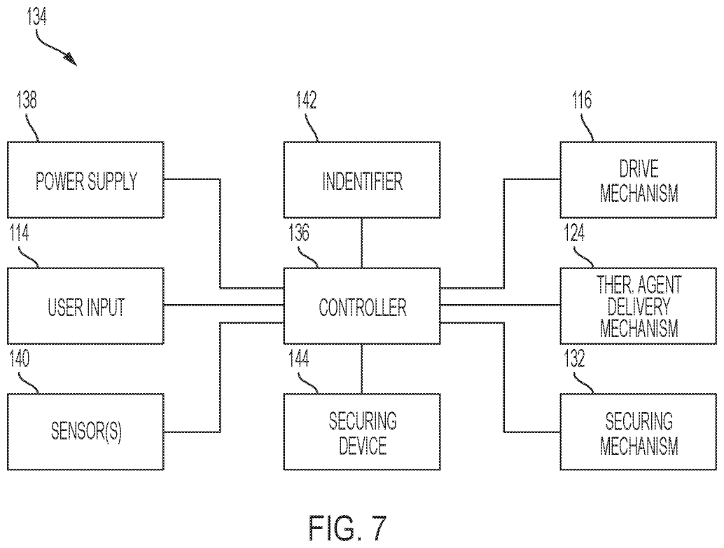

Referring to FIG. 7, the device 100 may also include a securing mechanism 132 for selectively securing the disposable portion 104 to the reusable portion 102. The securing mechanism 132 also facilitates easily attaching the disposable portion 104 to the reusable portion 102 and detaching the disposable portion 104 from the reusable portion 102. In addition to the above components, the therapeutic agent delivery device 100 also includes the electronics assembly 134 that facilitates operating the device 100 in the manners described herein. The electronics assembly 134 includes an electronic controller 136 that is operatively coupled to and receives power from the power supply 138, such as a battery. The electronic controller 136 also operatively couples to the user input 114 and one or more sensors 140. As described in further detail below, the sensors 140 may sense, for example, actuation of components of the device 100, positions of components of the device 100 relative to each other, and/or the position of the device 100 relative to a patient.

Components of the electronics assembly 134 may be carried by either portions 102, 104. In some embodiments and as described in further detail below, some components of the electronics assembly 134 are carried by the reusable portion 102 and some components are carried by the disposable portion 104 (both shown in FIGS. 1 and 2). For example, the disposable portion 104 may include an identifier 142 (for example, an RFID transmitter or EEPROM) to facilitate providing properties of the therapeutic agent 106 to the reusable portion 102. Such properties may include, for example, the type and/or volume of the therapeutic agent 106 carried by the syringe assembly 118. The reusable portion 102 may use the properties of the therapeutic agent 106 to determine, for example, if a patient associated with the reusable portion 102 is authorized to use, or has been prescribed, the therapeutic agent 106. As another example, a disposable portion may include securing device 144 that is operably coupled to the controller 136. The securing device 144 may initially inhibit the syringe 119 from moving from the stowed configuration to the deployed configuration, and the controller 136 may actuate the securing device 144 to permit the syringe 119 to move from the stowed configuration to the deployed configuration. In other embodiments, each of the components of the electronics assembly 134 is carried by the reusable portion 102. In some embodiments, the controller 136 is operatively coupled to one or more of the other components of the electronics assembly 134 by a wired connection. In some embodiments, the controller 136 is operatively coupled to one or more of the other components of the electronics assembly 134 by a wireless connection.

Electronics assembly 134 includes at least one processor that executes software and/or firmware stored in memory (not shown) of device 100. The software/firmware code contains instructions that, when executed by a processor, causes system controller 136 to perform the functions described herein. The at least one processor includes control logic/application operative to implement the operations described herein, including detecting device information, such as, for example, a dose delivered by the device based on a detected displacement or change in state of the sensed element relative to the housing/sensing element. Processor may be operative to store data indicative of the device information, such as, for example, the detected type, dose amount, time, and other data, into memory. Memory is any suitable computer readable medium that is accessible by the processor. Memory may be a single storage device or multiple storage devices, may be located internally or externally to the processor, and may include both volatile and non-volatile media. Exemplary memory includes random-access memory (RAM), read-only memory (ROM), electrically erasable programmable ROM (EEPROM), flash memory, a magnetic storage device, optical disk storage, or any other suitable medium which is configured to store data and which is accessible by a processor. The Electronics assembly 134 may include a display (not shown) through which at least portions of the device information or other information may be visually and/or audibly indicated. The display may be a LED indication or a LCD or other known forms of device displays. The Electronics assembly 134 may include a communication module (not shown) that is further operative to wirelessly transmit and/or receive a signal representative of the device information to a paired remote electronic device, such as a user's smartphone or cloud or server, over a Bluetooth low energy (BLE) or other suitable short or long range wireless communication protocol, such as, for example, near-field communication (NFC), WIFI, or cellular network. Similar to the device 100, the remote electronic device (not shown) includes a processor, memory, communication device, and also may include a display for user interface. Remote electronic device may includes a mobile device, such as a smartphone. Alternatively, any suitable computing device may be used, including but not limited to a laptop, desktop, tablet, or server computer, for example.

With reference now to FIG. 8, a transverse cross section of various of the internal components of the drive mechanism 116 and the shuttle system 109 is shown. Specifically, the carriage 146, also referred to as the motor shuttle, that is translatably carried in the housing 112′ (FIG. 4) or the housing 112 (FIG. 5) of the reusable portion 102. The carriage 146 carries the actuator 148 that operatively couples to the electronic controller 136. The actuator 148 may be a rotary actuator, more specifically an electric motor, that drivably couples to a transmission or speed reducer.

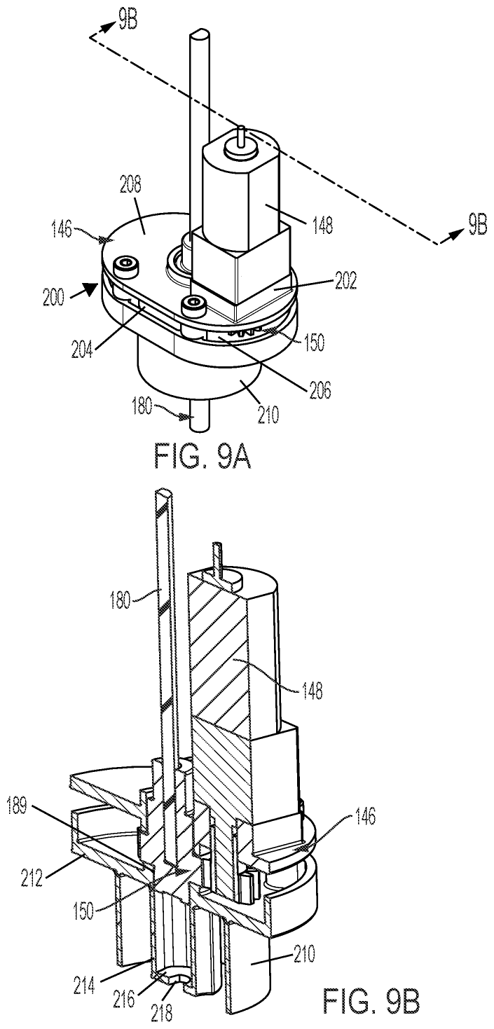

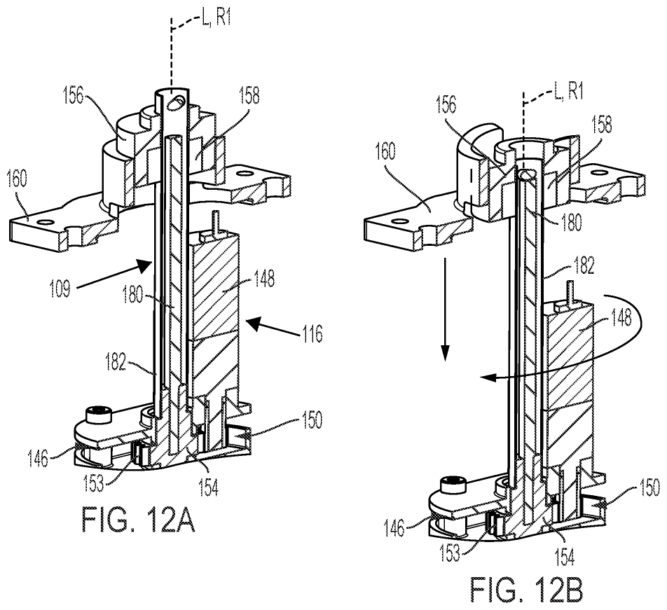

With additional reference to FIGS. 9A-10B, the actuator 148 drivably couples to the gear train 150. The gear train 150 may comprise a single gear, or as shown, a first gear 152 that drivably couples to a second gear 153 which is rotationally fixed to a nut 154, also referred to herein as a drive nut, such that movement of the first gear 152 and the second gear 153 causes rotation of the nut 154 about a rotation axis R1 (FIG. 12A) that is substantially parallel to the longitudinal axis L of the device 100 (that is, parallel ±10 degrees). Axis L may be coaxial with the axis R1. The nut 154 is operatively coupled to the lead screw 180, also referred to as a drive screw, that extends along the longitudinal axis L (FIG. 1) within the device 100. The gear train 150 is positioned at least partially within the carriage 146, and the carriage 146 comprises an axial through-opening 189 aligned with a passageway 190, also referred to as a bore, of the nut 154 for receiving the lead screw 180 and a coupling tube 182. As illustrated best in FIG. 10B, the passageway 190 of the nut 154 is threaded such that there is threaded engagement between the nut 154 and the lead screw 180 when therapeutic agent delivery device 100 is assembled. The threaded engagement between the nut 154 and the lead screw 180 is configured such that the nut 154 is axially coupled with the lead screw 180 in both axial directions (i.e., proximally and distally). In this way, rotation of the nut 154 in either a clockwise or counterclockwise direction will cause displacement of the lead screw 180 relative to the nut 154 in either the proximal or distal direction as the lead screw 180 moves proximally and/or distally within the passageway 190. In other words, rotation of the nut 154 causes axial movement of the lead screw 180 since the nut 154, the coupling tube 182 and the clutch 158 are rotationally fixed to one another. More specifically, rotation of the nut 154 in the clockwise direction causes displacement of the lead screw 180 in the first direction, such as the proximal direction, along the longitudinal axis L, while rotation of the nut 154 in the counterclockwise direction causes displacement of the lead screw 180 in the second direction, opposite the first direction, such as the distal direction along the longitudinal axis L.

The displacement of the lead screw 180 in the distal direction allows for the lead screw 180 to engage the plunger 126 (FIG. 6) and thus the interaction of the gear train 150, the actuator 148, the nut 154, and the lead screw 180 may act as a plunger or stopper drive system. Further, the coupling tube 182 surrounds at least a portion of the lead screw 180 and extends longitudinally from the carriage 146 in a proximal direction to the follower 156, as shown in FIG. 8. More specifically, the coupling tube 182 couples with the follower 156 by receiving a post 181 extending through the coupling tube 182 and above the follower 156. In this way, in some embodiments, rotation of the lead screw 180 and the coupling tube 182 causes rotation of the follower 156 and a clutch 158. In other words, the nut 154, the coupling tube 182, the lead screw 180, the follower 156, the clutch 158, and the guide 160 are in a coaxial relationship, as will be described further herein.

As shown in FIGS. 9A-9B, the carriage 146 includes a main body 200 with an actuator mount portion 202 that is configured for receiving the mounting of the actuator 148. In one example, the mount portion 202 is configured to allow the actuator 148 to be oriented axially along an axis that is offset and in parallel with the longitudinal axis L. The carriage 146 may include a gear housing portion 204. The gear housing portion 204 may define a cavity between a carriage proximal surface 206 and a plate 208 that attaches to the carriage proximal surface 206 in an axially spaced relationship to form the main body, where the gears and the nut are disposed and allow to rotate. The carriage 146 may also include an at least partially cylindrical shroud 210 extending distally from a distal surface 212 of the main body of the carriage and defined about the longitudinal axis L. The carriage 146 may also include an at least partially cylindrical push member 214 extending distally from the distal surface 212 of the main body and defined about the longitudinal axis L. The push member 214 is shown to have a smaller diameter than the shroud 210 thereby placing the push member within the shroud. The push member 214 may include a distal end 216 at least partially closed to define a push surface to engage or engaged with and move the proximal end of the syringe, and specifically the flange 111 of the syringe 119. In one example, a spacer 217 may be disposed inside of the proximal portion 143 of the assembly housing 141, along the proximal surface of the flange 111 such that the push surface of the push member 214 engages the spacer directly, rather than the flange 111. The spacer transmits forces from the push member 214 to the syringe flange 111, but may shield the flange from direct impact forces of the push member 214 that could cause damage to the syringe. The material of spacer 217 may be force dampening properties, and some example materials may have a durometer of 0-70 (Shore A).

Other material examples for the spacer 217, include, but are not limited to, elastomer, polyvinyl chloride, urethane, polytetrafluoroethylene, or other plastics. Spacer 217 is shown have an annular shape, having an inner cross-sectional area sized to inhibit the push member 214 from passing through, and an outer cross-sectional area sized no larger than the inner cross-sectional area of the proximal portion 143 of the assembly housing 141. Engagement between the push member 214 and the spacer 217, sitting on top of the syringe flange 111 is shown in FIG. 6. The push member 214 may be configured to have a portion inserted distally inside the syringe beyond the flange. A lead screw opening 218 may be defined centrally in the distal end 216 along the axis L, through which the lead screw 180 can move relative to the push member.

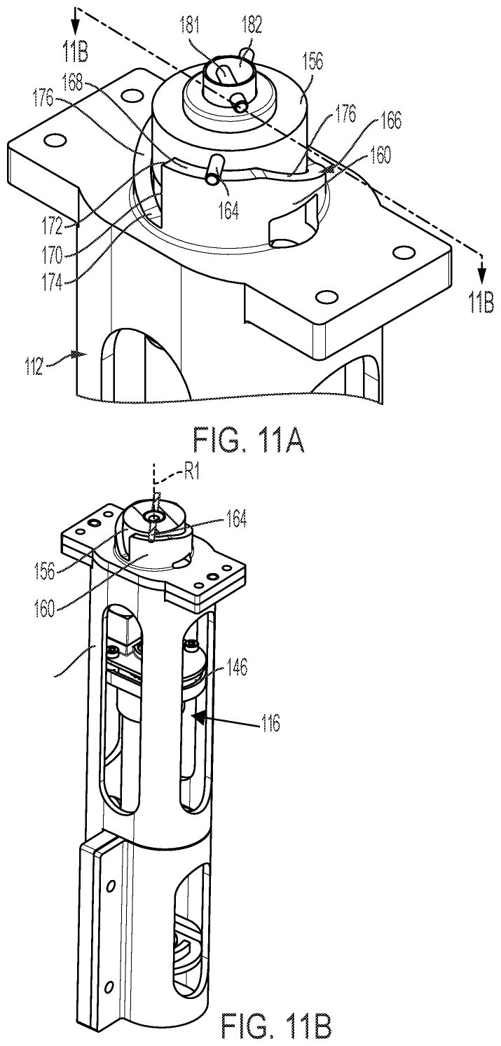

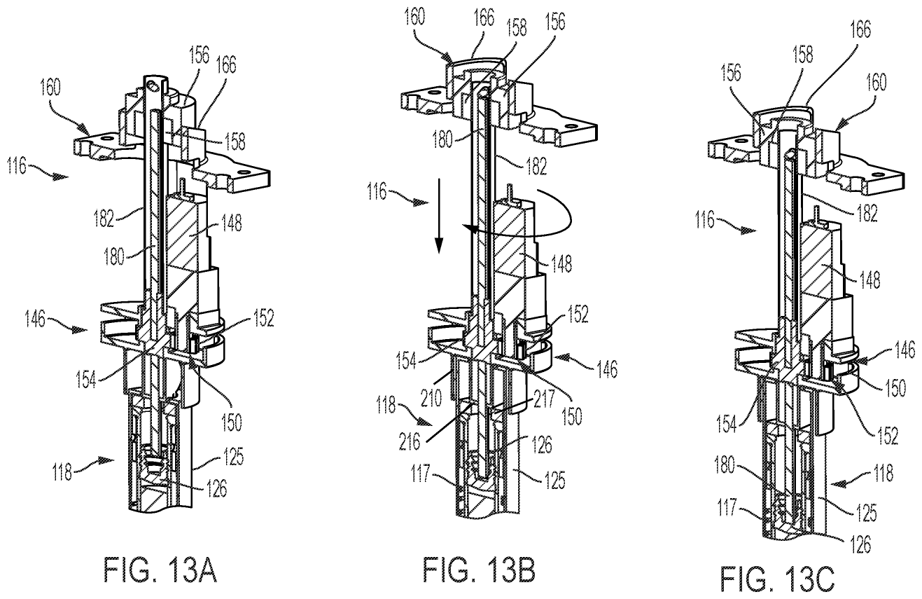

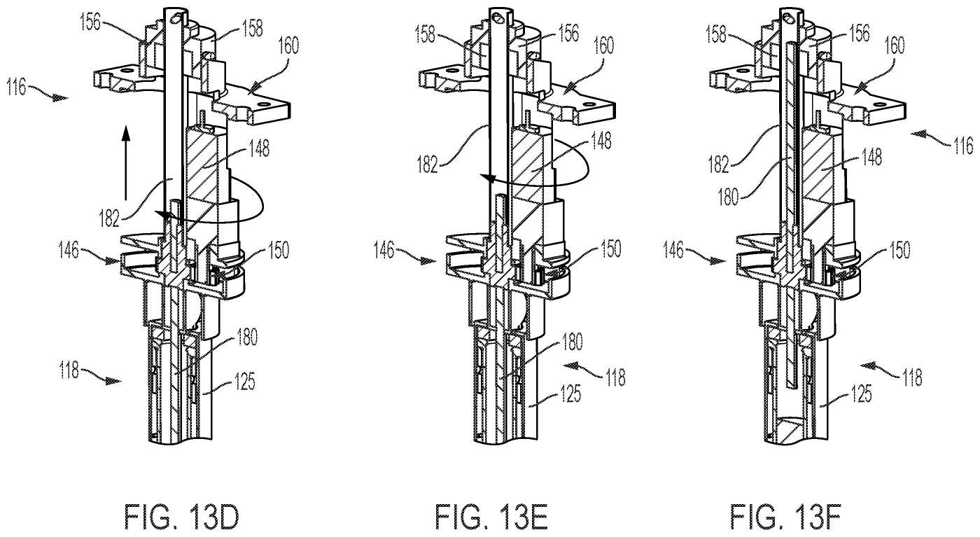

With reference to FIGS. 11A-11B, the drive mechanism 116 comprises the clutch 158 (FIG. 8) positioned within the follower 156. As previously described, the clutch 158 is fixed to the follower 156 and as such, the rotation of the coupling tube 182, due to the actuator driven rotation of the nut 154, causes rotation of the follower 156 in a first direction via the clutch 158. Further, the clutch 158 is axially and rotatably fixed with the follower 156 such that rotational movement of the clutch 158 cause rotation of the follower 156, and axial movement of the clutch 158 causes axial movement of the follower 156. More specifically, the clutch 158 may be a one-way clutch. In this way, the clutch 158 only rotates in one of the rotational directions, such as, for example, in a counterclockwise direction, and only counterclockwise rotation of the coupling tube 182 driven by the actuator 148 causes rotation and displacement of the follower 156 and the clutch 158. To this end, rotation of the coupling tube 182 in the opposite direction, that is the clockwise direction, would not result into rotation of the clutch 158 and thus the follower 156 does not rotate during this operational phase. This provides the advantage of the actuator 148 being capable for providing the actuation required for movement of the syringe 119 into the deployed configuration and for causing expulsion of the therapeutic agent from the needle 120, as will be described further with reference to FIGS. 13A-13F. However, various other embodiments of a clutch may be used. For example, a two-way clutch may be used and various other mechanisms for limiting the directionality of the rotation may be incorporated.

With reference still to FIGS. 11A-11B, the follower 156 movably couples to the guide 160, also referred to herein as a cam ramp, and the guide 160 is fixed relative to the housing 112 or 112′ of the reusable portion 102. The insertion biasing member 159 is shown positioned between the follower 156 and the housing 112 (see FIG. 6). Such member 159 in its expanded configuration can engage between the end cap of the housing 112 and the follower 156 or short of engagement with the housing as shown. While illustrated as a spring, the insertion biasing member 159 may take various other forms, for example a pulley, a cord, or any other applicable biasing means. The insertion biasing member 159 (FIGS. 4 and 6) coupled to the follower 156 is configured to urge the follower 156 distally and into engagement with the guide 160. Generally, the follower 156 and the guide 160 include features that facilitate movement of the follower 156 relative to the guide 160 as the follower 156 rotates about the rotation axis R1. The follower 156 can include one or more radial protrusions that movably engage a track defined by the proximal facing surface of the guide 160. In the embodiment shown, the follower 156 includes at least two radially-outwardly extending protrusions 164 that move along an angular track 166, or generally proximally-facing wall, defined by the guide 160 as the follower 156 is rotated by the actuator 148. The protrusions 164 simultaneously move along two similar sections, or halves, of the track 166. Referring specifically to FIG. 11A, each half of the track 166 includes a plateau portion 168 that couples to a cliff portion 170 at an edge 172, a valley portion 174 coupled to the cliff portion 170 opposite the plateau portion 168, and a slope portion 176 coupled to the valley portion 174 opposite the cliff portion 170. Each slope portion 176 also couples to the plateau portion 168 of the other half of the track 166.

In the illustrated embodiment of FIG. 11A, the various portions of the track 166 are as follows. The cliff portions 170 are substantially parallel to the longitudinal axis L of the device 100 (that is, parallel ±10 degrees). The plateau portions 168 and the valley portions 174 are substantially perpendicular to the longitudinal axis L of the device 100 (that is, perpendicular ±10 degrees). The slope portions 176 extend helically relative to the longitudinal axis L of the device 100.

In other embodiments, the follower 156 and/or the guide 160 may have different forms. For example, the track 166 could have a different shape. More specifically, the track 166 could include additional slope portions (not shown) instead of the cliff portions 170, and such slope portions could extend helically in the opposite directions as the slope portions 176. As another example, the follower 156 could include a different number of protrusions 164 and/or the guide 160 could include a track 166 with a different number of similar sections. For example, the follower 156 may include at least one protrusion 164. As yet another example, the follower 156 could include a track 166 that movably receives one or more protrusions 164 formed on the guide 160.

In the illustrative embodiment of FIGS. 11A-11B, when the protrusions 164 of the follower 156 rotate about the axis R1 due to the activation of the actuator 148 as described herein such that the protrusions 164 are no longer positioned on the plateau portion 168 and are instead angularly positioned on the valley portions 174, the follower 156 is displaced distally relative to the guide 160 to a second position. More specifically, rotation of the clutch 158 in the clockwise, or first, direction caused by actuation of the actuator 148, causes rotation of the coupling tube 182 and causes rotation of the follower 156. The rotation of the follower 156 then causes the rotational movement of the protrusions 164 (FIG. 11B) about the axis R1 along the track 166, such that the protrusions 164 are positioned on the valley portions 174 with the aid of the insertion biasing member 159 (FIG. 6), defining the second position of the follower 156. The follower second position defines a second position of the shuttle system 109 and the drive mechanism 116. This actuation of the actuator 148 also causes slight retraction of the lead screw 180 via the nut 154, releasing the insertion biasing member 159 (FIG. 6) to urge with a spring force the distal displacement of the follower 156, coupling tube 182, the carriage 146 and other components of shuttle system 109 relative to the guide and the device housing. As such, distal displacement of the follower 156 and the clutch 158 causes distal displacement of the carriage and the drive mechanism 116, which includes at least the follower 156, clutch 158, the actuator 148, the gear train 150, the lead screw 180, the coupling tube 182, and the carriage 146. The distal displacement of the shuttle system 109 and drive mechanism 116 contributes to the translation of the syringe 119 relative the the syringe assembly housing 141 to move distally into the deployed configuration.

For example, FIG. 12A illustrates the shuttle system 109 and the drive mechanism 116 in the first position. More specifically, the first position is defined by the protrusions 164 (FIG. 11A) of the follower 156 positioned on the plateau portion such that the follower 156 is held at an upwards or more proximal position relative to the proximal end 108 of the therapeutic agent delivery device 100. Rotation of the clutch 158 as described with reference to FIGS. 11A and 11B, causes rotation of the follower 156 about the axis R1. This resulting position is shown in FIG. 12B. Illustratively, the clutch 158 and the follower 156 are positioned further distally along the longitudinal axis L relative to the positioning of FIG. 12A. In addition to the displacement of the clutch 158 and the follower 156, the carriage 146 carrying the gear train 150 is also displaced distally.

With reference now to FIGS. 13A-13F, the operation of the therapeutic agent delivery device 100 will be detailed further. FIG. 13A illustrates the therapeutic agent delivery device 100 in a starting position, wherein the shuttle system 109 and the drive mechanism 116 are in the first position prior to any displacement of the shuttle system and the drive mechanism, and more specifically, prior to displacement of the follower 156. The insertion biasing member 159 (FIG. 6) is in the loaded configuration. The retraction biasing member 117 is in the unloaded configuration (FIG. 13B). The actuator drive shaft of actuator 148 may then be actuated in a clockwise direction, which causes rotation of the first gear 152, the nut 154 and thus the coupling tube 182 in their respective rotational directions. In this way, the rotation of the follower 156 about the axis R1 causes protrusions 164 (FIG. 11A) to drop to the valley portion 174 (FIG. 11A) of the track 166, as discussed with reference to FIGS. 11A and 11B. As such, the shuttle system 109 and the corresponding components of the drive mechanism 116 displaces distally relative to the proximal end 108 of the therapeutic agent delivery device 100, as illustrated in FIG. 13B. Additionally, this causes rotation of the lead screw 180, releasing the insertion biasing member 159 (FIG. 6), to further aid in the distal displacement of the syringe 119. During this movement, the force of the insertion biasing member 159 is greater than the force of the retraction biasing member 117 to thereby load the retraction biasing member 117, making it ready for the retraction operational phase subsequently. During distal movement of the shuttle system 109 relative to the syringe assembly housing 141 of the syringe assembly 118, shroud 210 of the carriage fits over the proximal end of the syringe assembly 118. The distal end 216 of the push member extends into an opening 118a (FIG. 4) disposed in the proximal end of the proximal portion 143 of the syringe assembly 118 to engage the spacer 217 on top of the syringe flange 111 and drive the syringe 119 to move distally relative to the syringe assembly housing. This positioning of FIG. 13B defines the second position of the shuttle system 109 and the drive mechanism 116, and defines the deployed position of the syringe assembly 118 after distal displacement of the syringe 119. More specifically, after distal displacement of the syringe 119, the syringe 119 is moved distally relative to the syringe assembly housing 141.

With reference to FIG. 13C, the actuator shaft of actuator 148 may then be actuated in the counterclockwise direction, or the second direction, drivably engaging and rotating the components of the gear train 150. As the lead screw 180 is axially coupled relative to the nut 154 due to the threaded engagement between the nut 154 and the lead screw 180, the rotation of the nut 154 in the clockwise direction causes rotation and axial displacement of the lead screw 180 distally and along the longitudinal axis L. Due to the clutch 158 being a one-way clutch, the movement of the actuator 148 in the counterclockwise direction fails to cause any rotation or axial displacement of the clutch 158 and the follower 156. In this way, the actuator 148 causes distal displacement of the lead screw 180 without any displacement of the carriage 146. As previously described, the distal displacement of the lead screw 180 causes the lead screw 180 to engage with and drivably move the plunger 126 of the syringe assembly 118 while in its deployed position. This subsequently causes the expulsion of the therapeutic agent 106 from the syringe assembly 118. There can be a full expulsion of the agent 106 in a single operation or there can be multiple deliveries of the agent 106 in different operations.

With reference to FIG. 13D, once the therapeutic agent 106 is fully expelled, the actuator shaft of the actuator 148 is actuated once again in the clockwise direction. This causes the retraction of the lead screw 180 as the nut 154 is threadedly engaged with the nut 154 in both axial directions (i.e., coupled proximally and distally). Additionally, the clutch 158 is rotated due to the clockwise rotation of the coupling tube 182. Thus, the follower 156 rotates about the axis R1 due to its coupling with the clutch and climbs back up the track 166 of the guide 160 until the protrusions 164 are once again rotationally positioned on the plateau portion 168, which defines the first position of the follower 156 and the first position of the drive mechanism 116. Additionally, during the movement of the clutch 158 and the follower 156, and the carriage 146 in the proximal direction along the longitudinal axis L, the syringe 119 is proximally displaced and the needle 120 is retracted back into the retracted position within the syringe assembly 118 and the therapeutic agent delivery device 100. Additionally, this causes the releasing of the retraction biasing member 117 (FIG. 13B), to further aid in the proximal displacement of the syringe 119 within the syringe assembly 118 with a spring force.

With reference to FIG. 13E, the various internal components of the therapeutic agent delivery device 100 are illustrated with the drive mechanism 116 positioned in the first position, and the lead screw 180 still distally displaced. Stated differently, the needle 120 has been retracted, however the lead screw 180 has not been fully retracted. Additionally, in this configuration, a lockout feature (not shown) may be implemented to inhibit the needle 120 from being extended out of the distal end 110 of the therapeutic agent delivery device 100 for a second time.

Actuation of the actuator shaft of the actuator 148 continues in the clockwise direction until the lead screw 180 is fully retracted back into the original position and the follower 156 is in the first position, such that the syringe assembly 118 is no longer in the deployed configuration. Additionally, this causes the loading of the insertion biasing member 159 (FIG. 6) for the next use. This positioning is illustrated in FIG. 13F. Once in this position, the actuation and rotation of the actuator 148 may be terminated. As previously described, the syringe assembly 118 as the disposable portion 104 may then be detached from the remainder of the device 100 and a new disposable portion loaded into the device 100. The device 100 may then be used for repeated dispensing of the therapeutic agent in accordance with the above described method.

As described previously, the use of the one way clutch 158 in combination with the remaining components of the drive mechanism 116 allows for the use of the therapeutic agent delivery device 100 with one single motor, as opposed to at least two motors, which may be required in conventional designs of a medication delivery device. For example, rather than requiring a first motor for placing the syringe assembly 118 in the deployed configuration and a second motor for causing the displacement of the lead screw and displacement of the plunger to expel the therapeutic agent, the present disclosure allows for the use of one motor to accomplish both functions. This provides various advantages, for example, at least the ability to reduce the space required within the therapeutic agent delivery device 100 for the internal components. In this way, the size of the therapeutic agent delivery device 100 may be optimized which can increase portability and convenience of the device. Further, through reducing the amount of motors required, the manufacturing costs may be reduced and the easy of manufacturing and/or assembly may be increased.

While this invention has been shown and described as having preferred embodiments, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

Various aspects are described in this disclosure, which include, but are not limited to, the following aspects:

1. A therapeutic agent delivery device having a proximal end and a distal end, including: a syringe assembly including a syringe having a plunger; a shuttle system coupled to the syringe: a drive mechanism including a lead screw, the lead screw having a distal end configured to engage the plunger; and a controller configured to: activate the drive mechanism to rotate the lead screw in a first direction to a position to facilitate movement of the shuttle system distally relative to the syringe assembly and movement of the syringe distally relative to the syringe assembly from a first position to a second position to extend a needle from the distal end of the therapeutic agent delivery device; activate the drive mechanism to rotate the lead screw in a second direction, the distal end of the lead screw driving the plunger of the syringe in the second position for therapeutic agent delivery; activate the drive mechanism to rotate the lead screw in the first direction after rotation of the said lead screw in the second direction to retract the distal end of the lead screw from the plunger and to facilitate movement of the shuttle system proximally and the syringe proximally relative to the syringe assembly from the second position toward the first position; and end activation of the drive mechanism to stop rotation of the lead screw after proximal movement of the shuttle system and the syringe.

2. The therapeutic agent delivery device of aspect 1, the shuttle system includes a spring, wherein rotation of the lead screw in the first direction releases the spring to drive the syringe distally.

3. The therapeutic agent delivery device of any one of aspects 1-2, wherein the shuttle system further includes a clutch operatively coupled to the lead screw, a cam follower coupled to the clutch, and a guide positioned radially relative to the cam follower.

4. The therapeutic agent delivery device of aspect 3, the drive mechanism further includes an actuator, a drive nut operably coupled with the actuator, and the shuttle system includes a coupling tube coupled between the drive nut and the clutch, wherein the drive nut, the coupling tube and the clutch are rotationally fixed relative to one another.

5. The therapeutic agent delivery device of aspect 4, wherein the drive nut includes a threaded bore for engaging along an outside of the lead screw, wherein in response to rotation of the drive nut the lead screw is axially movable.

6. The therapeutic agent delivery device of aspect 4, wherein the drive nut, the coupling tube, the lead screw, the cam follower, and the guide are in a coaxial relationship.

7. The therapeutic agent delivery device of any one of aspects 4-6, wherein the clutch is rotatably and axially fixed to the cam follower, wherein rotation of the clutch in the first direction causes rotation of the cam follower in the first direction and rotation of the clutch in the second direction does not cause rotation of the cam follower in the second direction.

8. The therapeutic agent delivery device of any one of aspects 4-7, wherein the guide includes a plateau portion and a valley portion disposed more distal than the plateau portion, and wherein the shuttle system includes a first position defined by a protrusion of the cam follower positioned on the plateau portion of the guide and a second position defined by the protrusion of the cam follower positioned on the valley portion of the guide.

9. The therapeutic agent delivery device of any one of aspects 4-8, wherein the shuttle system includes a carriage translatable within a housing of the therapeutic agent delivery device, the drive mechanism includes a gear train, the carriage carrying the actuator and the gear train coupled between the actuator and the drive nut, and the drive nut coupled to the clutch and the cam follower via the coupling tube.

10. The therapeutic agent delivery device aspect 9, wherein the carriage includes a push element engageable with the syringe.

11. The therapeutic agent delivery device of aspect 10, wherein the actuator is mounted on the carriage such that the actuator extends along a longitudinal axis of the lead screw.

12. The therapeutic agent delivery device of aspect 10, wherein the carriage includes a shroud extending distally, the shroud disposed radially outside of the push element.

13. A plunger drive and syringe movement system including, a cam follower, a cam ramp, a one-way clutch coupled to the cam follower, and a carriage coupled to a syringe of a syringe assembly; and a lead screw that is operatively coupled to the cam follower, the lead screw including a distal end to engage a plunger within the syringe, and a motor operatively coupled to the lead screw to drive the lead screw in a first direction and a second direction, wherein a drive of the lead screw in the first direction facilitates rotation of the cam follower to a position relative to the cam ramp to permit a displacement change to the carriage relative to the cam ramp; and wherein the drive of the lead screw in the second direction facilitates movement of the plunger distally to expel a therapeutic agent contained within the syringe.

14. The system of aspect 13, wherein the drive of the lead screw in the first direction causes proximal displacement of the lead screw, wherein the drive of the lead screw in the second direction causes distal displacement of the lead screw.

15. The system of any one of aspects 13-14, wherein the one-way clutch is configured such that rotation of the one-way clutch in a first direction allows rotation of the cam follower in the first direction, and rotation of the one-way clutch in a second direction inhibits rotation of the cam follower in the second direction.

16. The system of any one of aspects 13-15, further including a drive nut operatively coupled to the motor, and a coupling tube coupled between the one-way clutch and the drive nut, the coupling tube rotationally and axially fixed relative to the drive nut and the one-way clutch, the drive nut drivably engaged with the lead screw.

17. The system of aspect 16, wherein the drive nut and the motor is coupled to the carriage, the coupling tube and the lead screw extend axially from the carriage along a longitudinal axis, and the carriage further includes a push element extending distally along the longitudinal axis.

18. A method for delivering a therapeutic agent from a therapeutic agent delivery device, the therapeutic agent delivery device having a reusable assembly and a syringe assembly, the method including: providing the therapeutic agent delivery device in a rest configuration defined by a cam follower of the device being positioned in a first position on a plateau portion of a cam ramp; rotating the cam follower relative to the cam ramp to a second position within a valley portion of the cam ramp and thus displacing a shuttle assembly of the device and driving a syringe of said syringe assembly distally relative to the therapeutic agent delivery device to a deployed configuration; driving a lead screw distally along a longitudinal axis of the therapeutic delivery device, such that the lead screw displaces a plunger of the syringe in the deployed configuration to dispense a therapeutic agent.

19. The method of aspect 18, wherein driving the syringe distally includes positioning a needle of the syringe distally beyond a distal end of the therapeutic agent delivery device.

20. The method of aspect 19, wherein the method further includes the step of rotating the cam follower relative to the cam ramp from the second position to the first position and thus displacing the shuttle assembly and permitting the syringe of said syringe assembly to move proximally relative to the therapeutic agent delivery device from the deployed configuration to a stowed configuration.

21. The method of any one of aspects 18-20, wherein the reusable assembly includes a one-way clutch operatively coupled to the cam follower such that driving the lead screw distally does not cause rotation of the cam follower.

22. The method of any one of aspects 18-21, wherein the method further includes the step of detaching the syringe assembly from the therapeutic agent delivery device in order to allow for a replacement syringe assembly.

23. The device of any one of aspects 1-12, wherein the syringe assembly comprises a reservoir containing a therapeutic agent.

1. A therapeutic agent delivery device having a proximal end and a distal end, comprising:
a syringe assembly comprising a syringe having a plunger;
a shuttle system coupled to the syringe:
a drive mechanism comprising a lead screw, the lead screw having a distal end configured to engage the plunger; and
a controller configured to:
activate the drive mechanism to rotate the lead screw in a first direction to a position to facilitate movement of the shuttle system distally relative to the syringe assembly and movement of the syringe distally relative to the syringe assembly from a first position to a second position to extend a needle from the distal end of the therapeutic agent delivery device;
activate the drive mechanism to rotate the lead screw in a second direction, the distal end of the lead screw driving the plunger of the syringe in the second position for therapeutic agent delivery;
activate the drive mechanism to rotate the lead screw in the first direction after rotation of the said lead screw in the second direction to retract the distal end of the lead screw from the plunger and to facilitate movement of the shuttle system proximally and the syringe proximally relative to the syringe assembly from the second position toward the first position, and
end activation of the drive mechanism to stop rotation of the lead screw after proximal movement of the shuttle system and the syringe.

2. The therapeutic agent delivery device of claim 1, the shuttle system includes a spring, wherein rotation of the lead screw in the first direction releases the spring to drive the syringe distally.

3. The therapeutic agent delivery device of claim 1, wherein the shuttle system further includes a clutch operatively coupled to the lead screw, a cam follower coupled to the clutch, and a guide positioned radially relative to the cam follower.

4. The therapeutic agent delivery device of claim 3, the drive mechanism further comprises an actuator, a drive nut operably coupled with the actuator, and the shuttle system comprises a coupling tube coupled between the drive nut and the clutch, wherein the drive nut, the coupling tube and the clutch are rotationally fixed relative to one another.

5. The therapeutic agent delivery device of claim 4, wherein the drive nut comprises a threaded bore for engaging along an outside of the lead screw, wherein in response to rotation of the drive nut the lead screw is axially movable.

6. The therapeutic agent delivery device of claim 4, wherein the drive nut, the coupling tube, the lead screw, the cam follower, and the guide are in a coaxial relationship.

7. The therapeutic agent delivery device of claim 4, wherein the clutch is rotatably and axially fixed to the cam follower, wherein rotation of the clutch in the first direction causes rotation of the cam follower in the first direction and rotation of the clutch in the second direction does not cause rotation of the cam follower in the second direction.

8. The therapeutic agent delivery device of claim 4, wherein the guide comprises a plateau portion and a valley portion disposed more distal than the plateau portion, and wherein the shuttle system includes a first position defined by a protrusion of the cam follower positioned on the plateau portion of the guide and a second position defined by the protrusion of the cam follower positioned on the valley portion of the guide.

9. The therapeutic agent delivery device of claim 4, wherein the shuttle system comprises a carriage translatable within a housing of the therapeutic agent delivery device, the drive mechanism comprises a gear train, the carriage carrying the actuator and the gear train coupled between the actuator and the drive nut, and the drive nut coupled to the clutch and the cam follower via the coupling tube.

10. The therapeutic agent delivery device claim 9, wherein the carriage includes a push element engageable with the syringe.

11. The therapeutic agent delivery device of claim 10, wherein the actuator is mounted on the carriage such that the actuator extends along a longitudinal axis of the lead screw.

12. The therapeutic agent delivery device of claim 10, wherein the carriage includes a shroud extending distally, the shroud disposed radially outside of the push element.

13. A plunger drive and syringe movement system comprising,
a cam follower, a cam ramp, a one-way clutch coupled to the cam follower, and a carriage coupled to a syringe of a syringe assembly; and
a lead screw that is operatively coupled to the cam follower, the lead screw comprising a distal end to engage a plunger within the syringe, and a motor operatively coupled to the lead screw to drive the lead screw in a first direction and a second direction,
wherein a drive of the lead screw in the first direction facilitates rotation of the cam follower to a position relative to the cam ramp to permit a displacement change to the carriage relative to the cam ramp; and
wherein the drive of the lead screw in the second direction facilitates movement of the plunger distally to expel a therapeutic agent contained within the syringe.

14. The system of claim 13, wherein the drive of the lead screw in the first direction causes proximal displacement of the lead screw, wherein the drive of the lead screw in the second direction causes distal displacement of the lead screw.

15. The system of claim 13, wherein the one-way clutch is configured such that rotation of the one-way clutch in a first direction allows rotation of the cam follower in the first direction, and rotation of the one-way clutch in a second direction inhibits rotation of the cam follower in the second direction.

16. The system of claim 13, further comprising a drive nut operatively coupled to the motor, and a coupling tube coupled between the one-way clutch and the drive nut, the coupling tube rotationally and axially fixed relative to the drive nut and the one-way clutch, the drive nut drivably engaged with the lead screw.

17. The system of claim 16, wherein the drive nut and the motor is coupled to the carriage, the coupling tube and the lead screw extend axially from the carriage along a longitudinal axis, and the carriage further comprises a push element extending distally along the longitudinal axis.

18. (canceled)

19. (canceled)

20. (canceled)

21. (canceled)

22. (canceled)

23. The device of claim 1, wherein the syringe assembly comprises a reservoir containing a therapeutic agent.
